(19) United States
(12) Patent Application Publication
(10) Pub. No.: US 2014/0107382 A1
(43) Pub. Date: Apr. 17, 2014

(54) METHODS AND APPARATUSES FOR SEPARATING A LINEAR HEXANE STREAM FROM A HYDROCARBON FEED

(71) Applicant: UOP LLC, Des Plaines, IL (US)

(72) Inventors: David James Shecterle, Arlington Heights, IL (US); Mohamed Shakur, Hoffman Estates, IL (US)

(73) Assignee: UOP LLC, Des Plaines, IL (US)

(21) Appl. No.: 13/653,191

(22) Filed: Oct. 16, 2012

(51) Int. Cl. C07C 7/163; B01J 10/00

(52) U.S. Cl. 585/258; 422/187

(57) ABSTRACT

Methods of and apparatuses for separating a linear hexane stream from a hydrocarbon feed that includes unbranched C₄ to C₇ hydrocarbons are provided. In an embodiment, a method of separating a linear hexane stream from a hydrocarbon feed including unbranched C₄ to C₇ hydrocarbons includes isomerizing the hydrocarbon feed in the presence of hydrogen to produce an isomerized hydrocarbon stream that includes branched hydrocarbons and linear hexane. The isomerized hydrocarbon stream is separated into at least an isomerate product stream and a hexane-containing raffinate stream that includes linear hexane. The linear hexane stream is separated from at least a portion of the hexane-containing raffinate stream to produce the linear hexane stream and a hexane-depleted raffinate stream. The linear hexane stream is isolated as an independent product stream.

### TECHNICAL FIELD

The technical field generally relates to methods and apparatuses for separating a linear hexane stream from a hydrocarbon feed, and more particularly relates to methods and apparatuses for obtaining linear hexane streams that have low benzene content.

### BACKGROUND

Linear hexane, also known as normal hexane or n-hexane, is a valuable product that is used in industry for various applications. For example, linear hexane is useful as a solvent to extract cooking oils from seeds, for cleansing and degreasing a variety of items, and in textile manufacturing. Linear hexane is also useful as a solvent for production of biofuel from biomass. However, adequate separation of linear hexane from conventional hydrocarbon source streams that include linear hexane, other C6 paraffins, and benzene can be challenging, especially when the linear hexane has intended use for food-grade applications where components such as benzene are undesirable in even small amounts such as at parts-per-million (ppm) levels.

Hydrocarbon feed that includes linear hexane is commonly subject to refining processes for purposes of obtaining high-octane value products for including in fuel, such as gasoline. One example of a common refining process is isomerization of linear hydrocarbons in an isomerization stage in the presence of hydrogen and a reforming catalyst to form an isomerized hydrocarbon stream that has a higher content of branched hydrocarbons than the hydrocarbon feed. The branched hydrocarbons generally have a higher octane value than the corresponding linear hydrocarbons and are therefore valuable products for including in fuel. Equilibrium conditions during isomerization generally result in the presence of linear and cyclic hydrocarbons along with branched isomerates of the linear and cyclic hydrocarbons in the isomerized hydrocarbon stream. Because the linear and cyclic hydrocarbons decrease the octane value of the isomerized hydrocarbon stream and can be further isomerized, a variety of techniques are known for separating the branched hydrocarbons from unbranched hydrocarbons, with the unbranched hydrocarbons recycled in a recycle stream to be isomerized along with fresh hydrocarbon feed. For example, it is known to employ adsorption using an adsorbent bed that preferentially adsorbs linear hydrocarbons over branched or cyclic hydrocarbons to separate linear hydrocarbons from the isomerized hydrocarbon stream, desorbing the linear hydrocarbons from the adsorbent bed to form a recycle stream, and returning the recycle stream to an isomerization stage for isomerization along with fresh hydrocarbon feed. It is also known to employ fractionation using, for example, a deisohexanizer column to separate linear hexane, cyclic hydrocarbons, and monomethyl-branched pentane from hydrocarbons having a lower vapor pressure than linear hexane, cyclic hydrocarbons, and monomethyl-branched pentane. The linear hexane, cyclic hydrocarbons, and monomethyl-branched pentane are generally returned to the isomerization stage in the recycle stream. Recycling the linear hexane, cyclic hydrocarbons, and monomethyl-branched pentane to the isomerization stage maximizes process yield of the hydrocarbons that have higher octane values for including in gasoline. However, the recycle streams including the linear hexane, cyclic hydrocarbons, and/or monomethyl-branched pentane still contain a mix of compounds in high relative amounts. The recycle streams are therefore a desirable feed for the isomerization stage, but are not desirable end products themselves.

Accordingly, it is desirable to provide novel methods and apparatuses for separating a linear hexane stream from a hydrocarbon feed. It is also desirable to provide methods and apparatuses for separating a linear hexane stream from a hydrocarbon feed that enable high purity linear hexane to be obtained that has sufficiently low levels of benzene to enable use in food-grade applications. Furthermore, other desirable features and characteristics of the present invention will become apparent from the subsequent detailed description of the invention and the appended claims, taken in conjunction with the accompanying drawings and this background of the invention.

### BRIEF SUMMARY

Methods of and apparatuses for separating a linear hexane stream from a hydrocarbon feed that includes unbranched C₄ to C₇ hydrocarbons are provided. In an embodiment, a method of separating a linear hexane stream from a hydrocarbon feed including unbranched C₄ to C₇ hydrocarbons includes isomerizing the hydrocarbon feed in the presence of hydrogen to produce an isomerized hydrocarbon stream that includes branched hydrocarbons and linear hexane. The isomerized hydrocarbon stream is separated into at least an isomerate product stream that includes branched hydrocarbons and a hexane-containing raffinate stream that includes linear hexane. The linear hexane stream is separated from at least a portion of the hexane-containing raffinate stream to produce the linear hexane stream and a hexane-depleted raffinate stream. The linear hexane stream is isolated as an independent product stream.

In another embodiment, a method of separating a linear hexane stream from a hydrocarbon feed that includes unbranched C₄ to C₇ hydrocarbons includes isomerizing the hydrocarbon feed in the presence of hydrogen and in an isomerization stage that includes an isomerization catalyst to produce an isomerized hydrocarbon stream that includes branched hydrocarbons and linear hexane. The isomerized hydrocarbon stream is separated into at least an isomerate product stream that includes branched hydrocarbons and a hexane-containing raffinate stream that includes linear hexane in an isomerate separation stage that is in fluid communication with the isomerization stage. The hexane-containing raffinate stream is split into a recycle stream and a recovery stream. The linear hexane stream is separated from the recovery stream in a hexane separation stage that is different from and in fluid communication with the isomerate separation stage to produce the linear hexane stream and a hexane-depleted raffinate stream. The recycle stream is returned to the isomerization stage, and the linear hexane stream is isolated as an independent product stream.

In another embodiment, an isomerization apparatus includes an isomerization unit for receiving a hydrocarbon feed that includes unbranched C₄ to C₇ hydrocarbons, and for isomerizing the hydrocarbon feed to produce an isomerized hydrocarbon stream. An isomerate separation unit is in fluid communication with the isomerization unit for receiving the isomerized hydrocarbon stream and for separating the isomerized hydrocarbon stream into an isomerate product stream and a hexane-containing raffinate stream. A linear hexane separation unit is in fluid communication with the hexane-containing raffinate stream from the isomerate separation unit for receiving the hexane-containing raffinate stream, for separating a linear hexane stream from at least a portion of the hexane-containing raffinate stream, and for isolating the linear hexane stream as an independent product stream.

### BRIEF DESCRIPTION OF THE DRAWINGS

The present invention will hereinafter be described in conjunction with the following drawing figures, wherein like numerals denote like elements, and wherein:

FIG. 1 is a schematic diagram of an embodiment of an isomerization apparatus including an isomerization zone, an isomerate separation zone, and a hexane separation zone;

FIG. 2 is a schematic diagram of another embodiment of an isomerization apparatus including an isomerization zone, an isomerate separation zone, and a hexane separation zone;

FIG. 3 is a schematic diagram of another embodiment of an isomerization apparatus including an isomerization zone, an isomerate separation zone including an adsorption unit, and a hexane separation zone including a fractionation unit;

FIG. 4 is a schematic diagram of another embodiment of an isomerization apparatus including an isomerization zone, an isomerate separation zone including an adsorption unit, and a hexane separation zone including a fractionation unit;

FIG. 5 is a schematic diagram of another embodiment of an isomerization apparatus including an isomerization zone, an isomerate separation zone including a fractionation unit, and a hexane separation zone including an adsorption unit;

FIG. 6 is a schematic diagram of another embodiment of an isomerization apparatus including an isomerization zone, an isomerate separation zone including a fractionation unit, and a hexane separation zone including a second fractionation unit; and

FIG. 7 is a schematic diagram of another embodiment of an isomerization apparatus including an isomerization zone, an isomerate separation zone including a fractionation unit, and a hexane separation zone including a second fractionation unit.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Methods and apparatuses for separating a linear hexane stream from a hydrocarbon feed are provided herein. The methods and apparatuses enable linear hexane streams to be obtained that contain sufficiently low levels of benzene, such as less than or equal to about 3 ppm, to enable use in food-grade applications. Such low levels of benzene are possible because the linear hexane stream is ultimately separated from a hexane-containing raffinate stream that is downstream of an isomerization stage, where any benzene that is present in the hydrocarbon feed is hydrogenated such that the presence of benzene in the hexane-containing raffinate stream is minimized. As used herein, the term “hexane-containing raffinate stream” refers to the hydrocarbon stream that has a linear hexane content and that is separated from the desired branched hydrocarbons that are produced in the isomerization stage. “Branched hydrocarbons,” as referred to herein, include saturated or unsaturated hydrocarbons having one or more tertiary or quaternary carbon atoms present therein, whereas “unbranched hydrocarbons” have no tertiary or quaternary carbon atoms present therein (i.e., all carbon atoms are bonded to either one or two other carbon atoms). In embodiments, the branched hydrocarbons and the unbranched hydrocarbons are branched alkanes and unbranched alkanes, respectively. Various separation techniques can then be employed to separate the linear hexane stream from the hexane-containing raffinate stream, as described in further detail below, without concern for benzene contamination of the linear hexane stream. The linear hexane stream is isolated as an independent product stream, separate from other isomerate products and raffinate streams produced in the methods and apparatuses described herein, for independent use in applications that are external to the methods and apparatuses that are described herein, such as for use in food-grade applications.

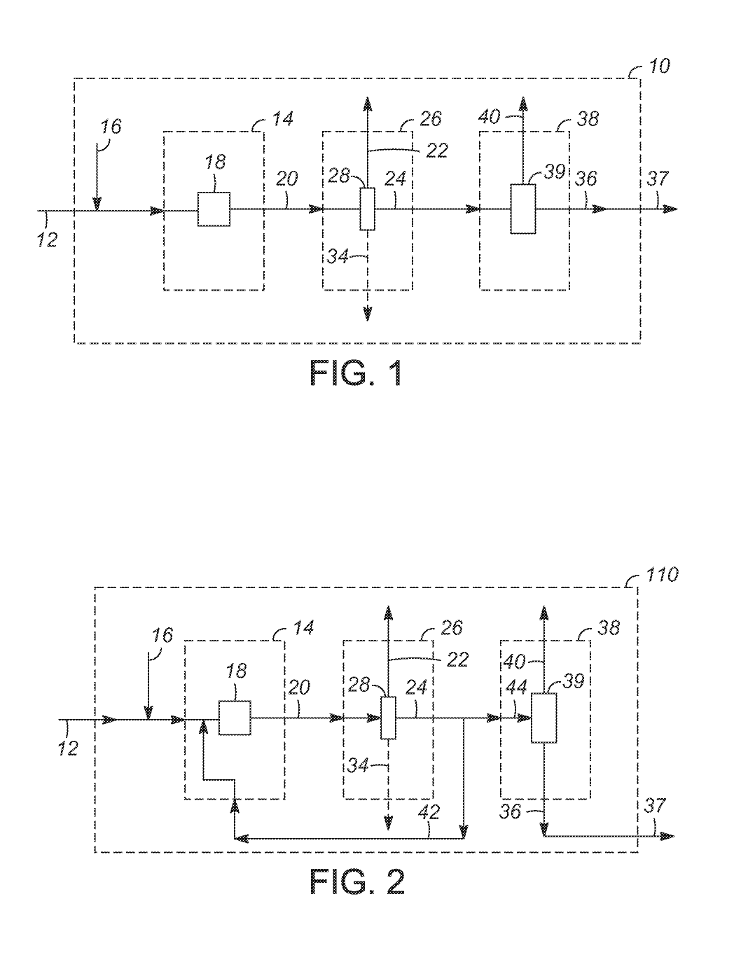

In an embodiment, and as shown in FIG. 1, a hydrocarbon feed 12 is provided in anticipation of isomerizing the hydrocarbon feed 12 in an isomerization stage 14 of an isomerization apparatus 10. Suitable hydrocarbon feeds 12 include those having hydrocarbon fractions that include unbranched C₄ to C₇ hydrocarbons, i.e., normal and cyclic paraffins. In an embodiment, a majority of hydrocarbons in the hydrocarbon feed 12 have 5 or 6 carbon atoms. The hydrocarbon feed 12, as referred to herein, represents a fresh source of hydrocarbons and excludes hydrocarbons from recycle streams or hexane-depleted raffinate streams that may be provided from unit operations that are disposed downstream of the isomerization stage 14, as described in further detail below. In an embodiment, the hydrocarbon feed 12 is rich in unbranched C₄ to C₇ hydrocarbons, meaning that the hydrocarbon feed 12 has at least 10 weight % of unbranched C₄ to C₇ hydrocarbons. Examples of suitable hydrocarbon feeds 12 include hydrocarbon streams having a majority of paraffins with from 4 to 6 carbon atoms, with only residual amounts of other hydrocarbons present. As used herein, “residual” refers to amounts that are at or below separation thresholds for the process referred to, and are typically amounts of less than or equal to about 1% by weight based upon the reference composition. Other useful hydrocarbon feeds 12 include natural gasoline, straight run naphtha, gas oil condensate, raffinates, reformate, field butanes, and straight run distillates having distillation end points of about 77° C. In other embodiments, the hydrocarbon feed 12 may also contain unsaturated hydrocarbons, hydrocarbons having more than 7 carbon atoms, and cyclic hydrocarbons.

As also shown in FIG. 1, hydrogen 16 is admixed with the hydrocarbon feed 12. In an embodiment, the hydrogen 16 is admixed with the hydrocarbon feed 12 prior to providing the hydrocarbon feed 12 into an isomerization unit 18 in the isomerization stage 14. However, it is to be appreciated that the hydrogen 16 can be separately provided into the isomerization unit 18 from the hydrocarbon feed 12. In an embodiment, the hydrogen 16 is provided in an amount that provides a hydrogen 16 to hydrocarbon ratio of less than or equal to about 0.10 in a resulting isomerized hydrocarbon stream 20 from the isomerization stage 14 when operating without hydrogen recycle, which provides sufficient excess hydrogen 16 to ensure that any unsaturated hydrocarbons that are introduced into the isomerization stage 14 are properly saturated. Although no net hydrogen 16 is consumed during isomerization of hydrocarbons in the isomerization stage 14, the isomerization stage 14 has a net consumption of hydrogen 16 that is associated with cracking, disproportionation, and olefin and aromatics saturation, and the excess hydrogen 16 ensures that sufficient amounts of hydrogen 16 are present in the isomerization stage 14 to enable the aforementioned reactions to occur.

Referring to FIG. 1, the hydrocarbon feed 12 is isomerized in the presence of hydrogen 16 in the isomerization stage 14 to produce the isomerized hydrocarbon stream 20 that includes branched hydrocarbons and linear hexane. The isomerization stage 14, as referred to herein, includes a unit or units of isomerization apparatus 10 where isomerization of unbranched hydrocarbons occurs and includes an isomerization catalyst for isomerizing the unbranched hydrocarbons that are introduced into the isomerization stage 14 to produce branched hydrocarbons, which are included in the isomerized hydrocarbon stream 20. Suitable isomerization catalysts are known in the art. The isomerization catalyst can be amorphous (e.g., based upon an amorphous inorganic oxide), crystalline (e.g., based upon a crystalline inorganic oxide), or a mixture of both. Isomerization catalyst containing a crystalline inorganic oxide generally contains an amorphous matrix or binder. The crystalline inorganic oxide can be a molecular sieve or a non-molecular sieve, or a mixture of a molecular sieve and a non-molecular sieve can be used. The molecular sieve can be zeolitic or non-zeolitic, or a mixture of a zeolite and a non-zeolite can be used. The isomerization catalyst may include platinum on mordenite, aluminum chloride on alumina, and platinum on sulfated or tungstated metal oxides such as zirconia. The isomerization catalyst may include a platinum group metal such as platinum on a chlorided alumina base, such as an anhydrous gamma-alumina A chloride component present in the isomerization catalyst, termed in the art “a combined chloride”, may be present in an amount from about 2% to about 10% by weight, such as from about 5% to about 10% by weight, based on the total weight of the isomerization catalyst.

In an embodiment and as shown in FIG. 1, the isomerization stage 14 includes an isomerization unit 18, with the isomerization catalyst (not shown) disposed therein, for receiving the hydrocarbon feed 12 and for isomerizing the hydrocarbon feed 12 to produce the isomerized hydrocarbon stream 20. While FIG. 1 only shows a single isomerization unit 18, it is to be appreciated that a plurality of isomerization units 18 may be employed in the isomerization stage 14. Isomerization units 18 that are known in the art and that can be employed in accordance with the methods and apparatuses 10 described herein include fixed-bed systems, moving-bed systems, fluidized-bed systems, or batch-type systems. The hydrocarbon feed 12 may be in the liquid phase, a mixed liquid-vapor phase, or a vapor phase when contacted with the isomerization catalyst. Suitable isomerization stages with a separator and a recycle gas compressor, without a separator and a recycle gas compressor, and without hydrogen recycle are all known in the art and are suitable for use in the methods and apparatuses 10 described herein.

Operating conditions within the isomerization stage 14 are selected to maximize the production of branched hydrocarbons from the unbranched hydrocarbons that are introduced therein. Operating conditions within the isomerization stage 14 are dependent upon various factors including, but not limited to, feed severity and catalyst type, and those of skill in the art are readily able to identify appropriate operating conditions within the isomerization stage 14 to maximize production of branched hydrocarbons. In an embodiment, when chlorided alumina and sulfated zirconia isomerization catalysts are used, a temperature within the isomerization stage 14 may be from about 90 to about 225° C. In another embodiment, when zeolitic isomerization catalysts are used, a temperature within the isomerization stage 14 may be from about 90 to about 290° C. The isomerization stage 14 may be maintained over a wide range of pressures, such as from about 100 kPa to about 10 MPa, or from about 0.5 MPa to about 4 MPa. A feed rate of all hydrocarbons to the isomerization stage 14 can also vary over a wide range, such as at liquid hourly space velocities of from about 0.2 to about 25 volumes of hydrocarbon per hour per volume of isomerization catalyst, such as from about 0.5 to 15 hf⁻¹.

The isomerized hydrocarbon stream 20 is separated into at least an isomerate product stream 22 that includes branched hydrocarbons and a hexane-containing raffinate stream 24 that includes linear hexane. The isomerate product stream 22 includes a higher content of branched hydrocarbons than the hexane-containing raffinate stream 24, and it is to be appreciated that the isomerized product stream 22 and the hexane-containing raffinate stream 24 can include additional chemical species other than branched hydrocarbons and linear hexane, respectively, as described in further detail below. In an embodiment and as shown in FIG. 1, the isomerized hydrocarbon stream 20 is separated in an isomerate separation stage 26 that is in fluid communication with the isomerization stage 14. As referred to herein, the isomerate separation stage 26 refers to a stage of the isomerization apparatus 10 that receives the isomerized hydrocarbon stream 20 and separates the isomerate product stream 22 therefrom. In an embodiment, the isomerized hydrocarbon stream 20 is separated into the isomerate product stream 22 and the hexane-containing raffinate stream 24 in the absence of intervening separation or supplementation stages between isomerizing the hydrocarbon feed 12 and separating the isomerized hydrocarbon stream 20. In other embodiments, additional separation or supplementation stages (not shown) may be disposed between the isomerization stage 14 and the isomerate separation stage 26.

Referring to FIG. 1, the isomerate separation stage 26 includes an isomerate separation unit 28 for separating the isomerized hydrocarbon stream 20. While FIG. 1 only shows a single isomerate separation unit 28, it is to be appreciated that a plurality of isomerate separation units 28 may be employed in the isomerate separation stage 26. The isomerate separation stage 26 can employ various types of separation units 28 and techniques to separate the isomerized hydrocarbon stream 20. In an embodiment, the isomerized hydrocarbon stream 20 further includes linear pentane and is separated by adsorbing linear pentane and linear hexane from the isomerized hydrocarbon stream 20 to form the hexane-containing raffinate stream 24 separate from the isomerate product stream 22. In embodiments, adsorption is conducted in the liquid phase or the vapor phase and can utilize any type of existing adsorption units such as a swing bed, simulated moving bed, or other schemes for contacting adsorbent material with the isomerized hydrocarbon stream 20 and desorbing the linear hydrocarbons from the adsorbent material with a desorbent material. The operating principles of the swing bed, moving bed, and simulated moving bed adsorption units are known in the art.

Virtually any adsorbent material that has capacity for the selective adsorption of the linear hydrocarbons can be employed in the adsorption units. Suitable adsorbents known in the art and commercially available include crystalline material including molecular sieves, activated carbons, activated clays, silica gels, activated aluminas and the like. Typically, the adsorbents contain the crystalline material dispersed in an amorphous inorganic matrix, or binder material, having channels and cavities therein that enable liquid access to the crystalline material. A variety of synthetic and naturally occurring binder materials are available such as metal oxides, clays, silicas, aluminas, silica-aluminas, silica-zirconias, silica thorias, silica-berylias, silica-titanias, silica-aluminas-thorias, silica-alumina-zirconias, mixtures of these and the like, and clay-type binders are suitable.

Examples of suitable desorbents that may be employed to desorb the linear hydrocarbons from the adsorbent material include C₄ to C₆ n-paraffins, e.g., n-butane, n-pentane, and n-hexane, which may be provided from a source that is external to the isomerate separation stage 26 or which may be provided by recovering desorbents within the isomerate separation stage 26. The desorbent stream may, in addition to the desorbent, contain up to 30% by weight, such as up to 5% by weight, of non-normal hydrocarbons such as branched alkanes and aromatics.

Adsorption conditions are not limited and may depend upon the phase within which adsorption is conducted. In an embodiment, adsorption is conducted in liquid phase in a temperature range of from about 60 to about 200° C., such as from about 100 to about 180° C., and a pressure sufficient to maintain liquid phase, such as from about atmospheric to about 3551 kPa, or from about atmospheric to about 1482 kPa. In an embodiment, desorption conditions include the same range of temperatures and pressures as used for adsorption conditions.

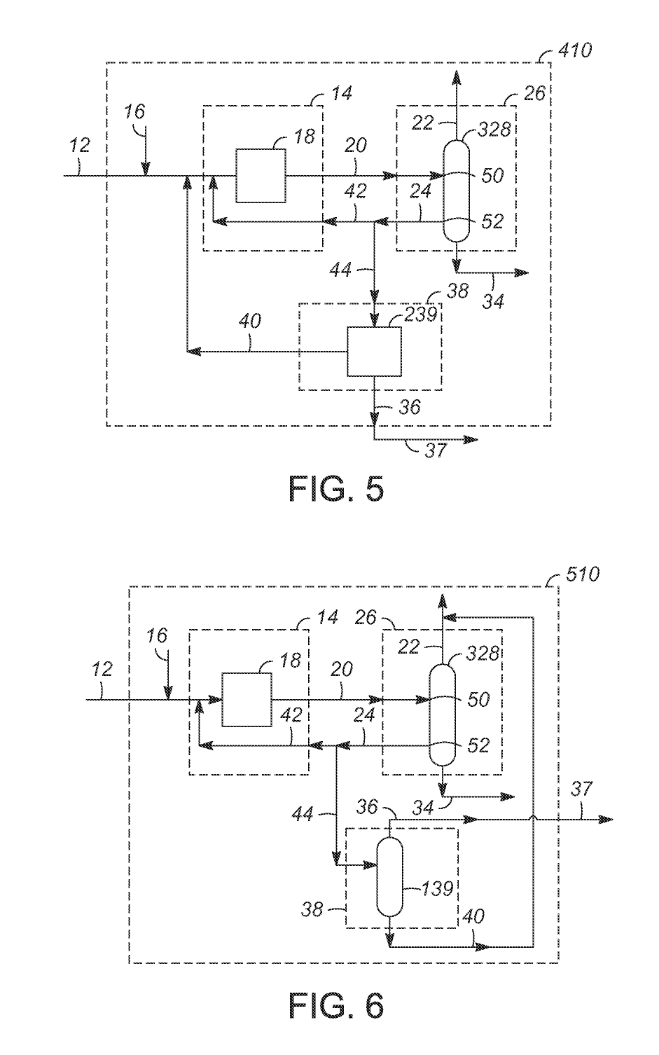

In another embodiment, referring momentarily to FIGS. 5 and 6, the isomerized hydrocarbon stream 20 is separated by fractionation in a fractionation unit 328 to produce the isomerate product stream 22 and the hexane-containing raffinate stream 24. In an embodiment, the fractionation unit 328 is a single fractionation column operated as a deisohexanizer. The general design and operation of such deisohexanizers is known to those of skill in the art. In the fractionation unit 328, the isomerized hydrocarbon stream 20 is fractionated into the isomerate product stream 22 in a fractionation overhead, a fractionation bottoms stream 34, and the hexane-containing raffinate stream 24 in a fractionation side draw. Additional features of the fractionation unit 328, as well as how fractionation is conducted in the fractionation unit 328, are described in further detail below.

The specific contents of the isomerate product stream 22 and the hexane-containing raffinate stream 24 depend upon the particular isomerate separation stage 26 that is employed. For example, when fractionation is employed in the isomerate separation stage 26, the isomerate product stream 22 is fractionated as a fractionation overhead and includes branched hydrocarbons having less than or equal to 6 carbon atoms and linear hydrocarbons having less than or equal to 5 carbon atoms, with the branched hydrocarbons being present in an amount that is higher than an amount of branched hydrocarbons in the hexane-containing raffinate stream 24. For example, in an embodiment, the branched hydrocarbons are present in the isomerate product stream 22 in an amount of at least 50% by weight based on the total weight of the isomerate product stream 22. In this embodiment, the hexane-containing raffinate stream 24 includes linear hexane, cyclic hydrocarbons, and monomethyl-branched pentane, with the linear hexane present in an amount of at least 10% by weight, such as from about 10% to about 20% by weight, based on the total weight of the hexane-containing raffinate stream 24. Also in this embodiment, the hexane-containing raffinate stream 24 is fractionated in the fractionation side draw, which may be in liquid phase. The fractionation bottoms stream 34 generally includes hydrocarbon isomerates that have a higher boiling point than the components in the hexane-containing raffinate stream 24, in addition to a content of components that are also present in the hexane-containing raffinate stream 24, and the fractionation bottoms stream 34 may be in liquid phase. As another example, when adsorption is employed in the isomerate separation stage 26, the isomerate product stream 22 is separated to include cyclic and branched hydrocarbons in the isomerate product stream 22, and the hexane-containing raffinate stream 24 is separated to include linear hydrocarbons, with only residual amounts of cyclic and branched hydrocarbons being present in the hexane-containing raffinate stream 24 in accordance with adsorption limits.

Referring to FIG. 1, a linear hexane stream 36 is separated from at least a portion of the hexane-containing raffinate stream 24, such as in a hexane separation stage 38 that is different from and in fluid communication with the isomerate separation stage 26, to produce the linear hexane stream 36 and a hexane-depleted raffinate stream 40. As referred to herein, the hexane separation stage 38 refers to the stage in the isomerization apparatus 10 that receives the hexane-containing raffinate stream 24 and produces the linear hexane stream 36 and the hexane-depleted raffinate stream 40. The hexane-depleted raffinate stream 40 has a lower content of linear hexane than the linear hexane stream 36. In an embodiment, the hexane separation stage 38 includes at least one linear hexane separation unit 39 that is in fluid communication with the hexane-containing raffinate stream 24 from the isomerate separation unit 28 for receiving the hexane-containing raffinate stream 24. As alluded to above, various separation techniques can be employed to separate the linear hexane stream 36 from the hexane-containing raffinate stream 24, and the particular separation technique may depend upon the contents of the hexane-containing raffinate stream 24. For example, when the hexane-containing raffinate stream 24 includes linear hexane, cyclic hydrocarbons, and monomethyl-branched pentane, adsorption can be employed to separate the linear hexane stream 36 from the cyclic hydrocarbons and monomethyl-branched pentane. The cyclic hydrocarbons and monomethyl-branched hydrocarbons are present in the hexane-depleted raffinate stream 40. Adsorption can be conducted in the same manner as described above with the linear hexane separation unit 39 being an adsorption unit. Residual amounts of cyclic hydrocarbons and monomethyl-branched pentane may remain in the linear hexane stream 36 in accordance with adsorption limitations. Alternatively, when the hexane-containing raffinate stream 24 is substantially free from cyclic hydrocarbons and monomethyl-branched pentane, but includes various linear hydrocarbons such as linear pentane and linear hexane, fractionation can be employed to separate the linear hexane from the linear pentane in the hexane-containing raffinate stream 24, in which case the linear hexane separation unit 39 is a second fractionation unit. In this embodiment, the linear pentane is recovered in an overhead pentane stream as the hexane-depleted raffinate stream 40 and the linear hexane stream 36 is recovered as a second fractionation bottoms stream. The hexane-depleted raffinate stream 40 can be recycled to the isomerization stage 14 or can be processed as an independent raffinate product stream (not shown). When the hexane-depleted raffinate stream 40 is recycled to the isomerization stage 14, the hydrocarbon feed 12 and the hexane-depleted raffinate stream 40 are isomerized in the isomerization stage 14.

The linear hexane stream 36 that is separated from the hexane-containing raffinate stream 24 is isolated as an independent product stream 37. In particular, the linear hexane stream 36 is provided as the independent product stream 37 resulting from the methods and apparatuses described herein, and is not recycled for use within the methods and apparatuses. However in embodiments, the linear hexane stream 36 is further purified to increase a concentration of linear hexane therein, depending upon particular application demands for purity of the linear hexane stream 36.

The linear hexane stream 36 that is produced in accordance with the methods described herein may have a hexane content of at least about 50% by weight based on the total weight of the linear hexane stream 36. In an embodiment, the hexane content of the linear hexane stream 36 is from about 52% by weight to about 99% by weight and has a benzene content of less than about 3 ppm, which is sufficiently pure to enable the linear hexane stream 36 to be utilized in food-grade applications. It is to be appreciated that higher hexane content in the linear hexane stream 36 can be achieved by employing the methods and apparatuses described herein, such as a hexane content of from about 65% to about 99% by weight, or from about 90% to about 99% by weight, based on the total weight of the linear hexane stream 36.

Various specific embodiments of methods and isomerization apparatuses for separating the linear hexane stream 36 from the hydrocarbon feed 12 will now be described with reference to FIGS. 2-7. As alluded to above, the linear hexane stream 36 is separated from at least a portion of the hexane-containing raffinate stream 24. In an embodiment and as shown in FIG. 2, the hydrocarbon feed 12 is isomerized in the presence of hydrogen 16 in the isomerization stage 14 of an isomerization apparatus 110, and the isomerized hydrocarbon stream 20 is separated into at least the isomerate product stream 22 and the hexane-containing raffinate stream 24 in the isomerate separation stage 26 in the manner described above. However, the hexane-containing raffinate stream 24 is split into a recycle stream 42 and a recovery stream 44, and the linear hexane stream 36 is separated from the recovery stream 44. As referred to herein, the “recovery” stream 44 refers to the stream from which linear hexane is ultimately recovered in the linear hexane stream 36, and the “recycle” stream 42 is ultimately recycled back to the isomerization stage 14. In this embodiment, the linear hexane stream 36 is separated from the recovery stream 44 in any manner as previously described above. Recycle streams 42 including unbranched hydrocarbons are common in existing isomerization units where unbranched hydrocarbons are separated from branched hydrocarbons for purposes of maximizing process yield. In this embodiment, the hydrocarbon feed 12 and the recycle stream 42 are isomerized in the isomerization stage 14. Although FIG. 2 shows the recycle stream 42 combined with the hydrocarbon feed 12 within the isomerization stage 14, it is to be appreciated that the methods and apparatuses described herein are not limited as to location at which the hydrocarbon feed 12 and recycle stream 42 are mixed, and the hydrocarbon feed 12 and recycle stream 42 can be mixed in the isomerization unit 18 itself.

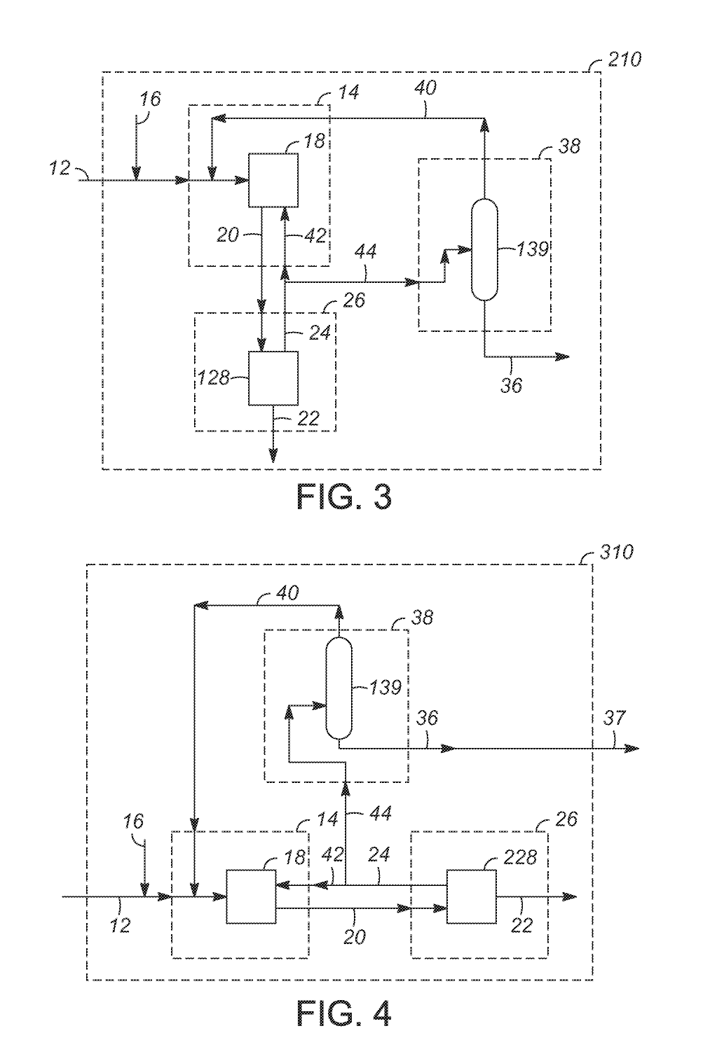

In other embodiments and as shown in FIGS. 3 and 4, the hydrocarbon feed 12 is isomerized in the presence of hydrogen 16 in the isomerization stage 14 of isomerization apparatuses 210, 310, as described above. However, in these particular embodiments, the isomerate separation stage 26 includes an adsorption unit 128, 228 that preferentially adsorbs linear hydrocarbons over branched and cyclic hydrocarbons, with FIG. 3 generally illustrating a liquid-phase adsorption unit 128 in the isomerization apparatus 210 and with FIG. 4 generally illustrating a vapor-phase adsorption unit 228 in the isomerization apparatus 310. The isomerized hydrocarbon stream 20 is separated by adsorbing linear hydrocarbons from the isomerized hydrocarbon stream 20 to form the hexane-containing raffinate stream 24 separate from the isomerate product stream 22. In the embodiments shown in FIGS. 3 and 4, the hexane-containing raffinate stream 24 is split into the recycle stream 42 and the recovery stream 44, as described above relative to the embodiment of FIG. 2, and the linear hexane stream 36 is separated from the recovery stream 44. In the embodiments of FIGS. 3 and 4, because the isomerized hydrocarbon stream 20 is separated through adsorption, the hexane-containing raffinate stream 24 is substantially free from cyclic hydrocarbons and monomethyl-branched pentane, although residual levels of cyclic and monomethyl-branched pentane may be present in accordance with adsorption limitations. The hexane-containing raffinate stream 24 includes various linear hydrocarbons such as linear pentane and linear hexane. The hexane separation stage 38 includes the second fractionation unit 139, which is effective for substantially separating the linear hexane from the linear pentane, with the linear pentane recovered in the overhead pentane stream as the hexane-depleted raffinate stream 40 and the linear hexane stream 36 recovered as a bottoms stream. It is to be appreciated that the linear hexane stream 36 may contain residual amounts of linear pentane due to fractionation inefficiencies. In the embodiments of FIGS. 3 and 4, the hexane-depleted raffinate stream 40 is returned to the isomerization stage 14, and the hydrocarbon feed 12. The hexane-depleted raffinate stream 40 and the recycle stream 42 are isomerized in the isomerization stage 14.

In other embodiments and as shown in FIGS. 5 and 6, the hydrocarbon feed 12 is isomerized in the presence of hydrogen 16 in the isomerization stage 14 of isomerization apparatuses 410, 510, as described above. However, in these particular embodiments, the isomerate separation stage 26 includes the fractionation unit 328 for fractionating the isomerized hydrocarbon stream 20 to produce the isomerate product stream 22 and the hexane-containing raffinate stream 24. More specifically, in the embodiments of FIGS. 5 and 6, the isomerized hydrocarbon stream 20 is fractionated to provide the isomerate product stream 22 in the fractionation overhead, a fractionation bottoms stream 34, and the hexane-containing raffinate stream 24 in a fractionation side draw. In this embodiment, the product isomerate stream 22 includes branched hydrocarbons having less than or equal to 6 carbon atoms and linear hydrocarbons having less than or equal to 5 carbon atoms. Specifically, the isomerate product stream 22 generally contains pentanes, and dimethylbutanes. The hexane-containing raffinate stream 24 that is separated in the fractionation side draw includes linear hexane, cyclic hydrocarbons, and monomethyl-branched pentane. Due to inefficiencies in separation, it is to be appreciated that residual amounts of various hydrocarbons can be present in the respective streams 22, 24, and that complete separation is rarely feasible. In an embodiment, the hexane-containing raffinate stream 24 includes from about 10 to 20% of linear hexane, based upon the total weight of the hexane-containing raffinate stream 24, with a balance of the hexane-containing raffinate stream being predominantly methylpentanes, cyclohexane, and methylcyclopentane, with residual amounts of dimethylbutanes and heptanes. Although not shown in FIGS. 5 and 6, the fractionation side draw is generally collected in a tray location below an input point 50 of the isomerized hydrocarbon stream 20. During fractionation in the fractionation unit 328, a cut point 52 for the fractionation side draw is generally maintained at a temperature below a boiling point of 2,3-dimethylbutane and cyclic hexane and above the boiling point of 2-methylpentane. Only a narrow boiling point difference separates 2,3-dimethylbutane and 2-methylpentane, and the location of the cut point 52 for the fractionation side draw enables separation of the 2,3-dimethylbutane and the 2-methylpentane, although a 2,3-dimethylbutane content generally remains in the hexane-containing raffinate stream 24 that is separated in the fractionation side draw. Hydrocarbons having a higher boiling point than linear and cyclic hexane and monomethyl pentanes are withdrawn from the fractionation unit 328 in the fractionation bottoms stream 34, although a cyclic hexane content is generally present in the fractionation bottoms stream 34 as well. Examples of hydrocarbons having a higher boiling point than linear and cyclic hexane and monomethyl pentanes include hydrocarbons having at least 7 carbon atoms.

In the embodiments shown in FIGS. 5 and 6, the hexane-containing raffinate stream 24 is split into the recycle stream 42 and the recovery stream 44, as described above relative to the embodiment of FIG. 2, and the linear hexane stream 36 is separated from the recovery stream 44. In the embodiment of FIG. 5, the linear hexane stream 36 is separated from the recovery stream 44 in the hexane separation stage 38 of the isomerization apparatus 410 that includes a second adsorption unit 239 to produce the linear hexane stream 36 and the hexane-depleted raffinate stream 40. Because linear pentane is substantially absent from the hexane-containing raffinate stream 24 in this embodiment, save for residual amounts, and because the second adsorption unit 239 preferentially adsorbs linear hydrocarbons over branched and cyclic hydrocarbons, separation of the linear hexane from the recovery stream 44 through adsorption is effective to obtain the linear hexane stream 36. The hexane-depleted raffinate stream 40 includes cyclic hydrocarbons and monomethyl-branched pentane that is separated from the linear hexane. In the embodiment of FIG. 5, the hexane-depleted raffinate stream 40 is returned to the isomerization stage 14. In this embodiment, the hexane-depleted raffinate stream 40, the hydrocarbon feed 12, and the recycle stream 42 are isomerized in the isomerization stage 14. In the embodiment of FIG. 6, the linear hexane is separated from the recovery stream 44 in the hexane separation stage 38 of the isomerization apparatus 510 that includes the second fractionation unit 139, which separates the recovery stream 44 into the linear hexane stream 36 as a fractionation overhead and the hexane-depleted raffinate stream 40 as a fractionation bottoms stream. As set forth above, in embodiments the 2,3-dimethylbutane content generally remains in the hexane-containing raffinate stream 24 that is separated in the fractionation side draw. After fractionation of the hexane-containing raffinate stream 24 in the hexane separation stage 38 of FIG. 6, 2,3-dimethyl butane is also generally present in the linear hexane stream 36, and additional fractionation may be conducted to further separate the linear hexane from the 2,3-dimethyl butane. In the embodiment shown in FIG. 6, the hexane-depleted raffinate stream 40 is combined with the isomerate product stream 22 that is separated from the isomerized hydrocarbon stream 20. The recycle stream 42 is returned to the isomerization stage 14, and the hydrocarbon feed 12 and the recycle stream 42 are isomerized in the isomerization stage 14.

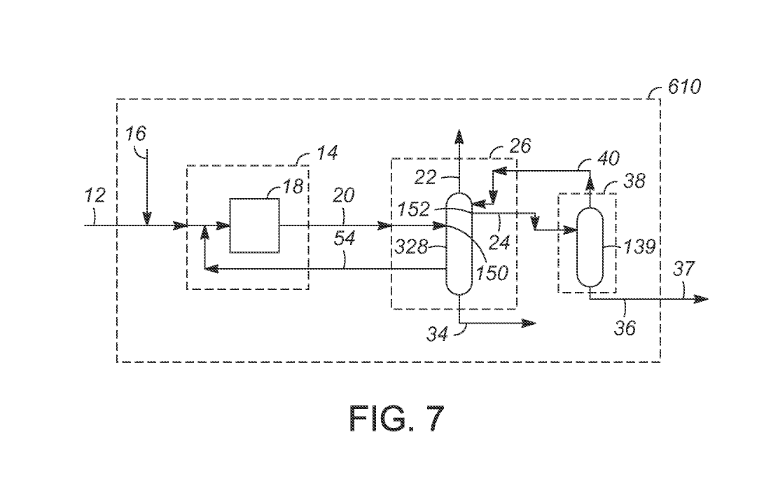

In another embodiment as shown in FIG. 7, the hydrocarbon feed 12 is isomerized in the presence of hydrogen 16 in the isomerization stage 14 of another isomerization apparatus 610, as described above, and the isomerized hydrocarbon stream 20 is fractionated to provide the isomerate product stream 22 in the fractionation overhead, a fractionation bottoms stream 34, and the hexane-containing raffinate stream 24 in the fractionation side draw as described above in the context of FIGS. 5 and 6. However, instead of splitting the hexane-containing raffinate stream 24, as is done in the isomerization apparatuses 410, 510 of FIGS. 5 and 6, the entire hexane-containing raffinate stream 24 is separated in the hexane separation stage 38 that includes the second fractionation unit 139 as described in the context of FIG. 6 to produce the linear hexane stream 36 and the hexane-depleted raffinate stream 40. Also in this embodiment, a cut point 152 for the fractionation side draw is above an input point 150 of the isomerized hydrocarbon stream 20 into the fractionation unit 328. Unlike the isomerization apparatus shown in FIG. 6, the linear hexane stream 36 is taken from the bottoms of the second fractionation unit 139 and the hexane-depleted raffinate stream 40 is taken as an overhead stream from the second fractionation unit 139. In this embodiment, the hexane-depleted raffinate stream 40 is returned to the fractionation unit 328 in the isomerate separation stage 26. As such, in this embodiment, the hexane-depleted raffinate stream 40 is fractionated in the presence of the isomerized hydrocarbon stream 20 in the fractionation unit 328 of the isomerate separation stage 26 to maximize the yield of 2,3-dimethylbutane in the isomerate product stream 22 in the fractionation overhead. Separate from the hexane-containing raffinate stream 24, a second recycle stream 54 including unbranched hydrocarbons can be fractionated in another fractionation side draw in the isomerate separation stage 26 separate from the hexane-containing raffinate stream 24. The second recycle stream 54 is returned to the isomerization stage 14 and the hydrocarbon feed 12 and the second recycle stream 54 are isomerized in the isomerization stage 14.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A method of separating a linear hexane stream from a hydrocarbon feed comprising unbranched C₄ to C₇ hydrocarbons, the method comprising:
isomerizing the hydrocarbon feed in the presence of hydrogen to produce an isomerized hydrocarbon stream comprising branched hydrocarbons and linear hexane;
separating the isomerized hydrocarbon stream into at least an isomerate product stream comprising branched hydrocarbons and a hexane-containing raffinate stream comprising linear hexane;
separating the linear hexane stream from at least a portion of the hexane-containing raffinate stream to produce the linear hexane stream and a hexane-depleted raffinate stream; and
isolating the linear hexane stream as an independent product stream;
wherein the linear hexane stream contains levels of benzene, less than or equal to about 3 ppm; and
wherein separating the linear hexane stream comprises splitting the hexane-containing raffinate stream into a recycle stream and a recovery stream, and wherein the linear hexane stream is separated from the recovery stream.

2. (canceled)

3. The method of claim 1, wherein isomerizing the hydrocarbon feed comprises isomerizing the hydrocarbon feed and the recycle stream.

4. The method of claim 1, wherein isomerizing the hydrocarbon feed comprises isomerizing the hydrocarbon feed and the hexane-depleted raffinate stream.

5. The method of claim 1, wherein the hexane-containing raffinate stream further comprises linear pentane, and wherein separating the isomerized hydrocarbon stream comprises adsorbing linear pentane and linear hexane from the isomerized hydrocarbon stream to form the hexane-containing raffinate stream separate from the isomerate product stream.

6. The method of claim 5, wherein separating the linear hexane stream from at least a portion of the hexane-containing raffinate stream comprises fractionating at least a portion of the hexane-containing raffinate stream to produce the linear hexane stream and the hexane-depleted raffinate stream.

7. The method of claim 1, wherein separating the isomerized hydrocarbon stream comprises fractionating the isomerized hydrocarbon stream to produce the isomerate product stream and the hexane-containing raffinate stream.

8. The method of claim 7, wherein fractionating the isomerized hydrocarbon stream comprises fractionating the isomerized hydrocarbon stream into the isomerate product stream in a fractionation overhead, a fractionation bottoms stream, and the hexane-containing raffinate stream in a fractionation side draw.

9. The method of claim 8, wherein separating the linear hexane stream from at least a portion of the hexane-containing raffinate stream comprises adsorbing linear hexane from the at least a portion of the hexane-containing raffinate stream to produce the linear hexane stream and the hexane-depleted raffinate stream.

10. The method of claim 8, wherein separating the linear hexane stream from at least a portion of the hexane-containing raffinate stream comprises fractionating at least a portion of the hexane-containing raffinate stream to produce the linear hexane stream and the hexane-depleted raffinate stream.

11. The method of claim 10, further comprising fractionating the hexane-depleted raffinate stream in the presence of the isomerized hydrocarbon stream.

12. The method of claim 11, wherein fractionating the isomerized hydrocarbon stream further comprises fractionating the isomerized hydrocarbon stream into a recycle stream comprising unbranched hydrocarbons in another fractionation side draw separate from the hexane-containing raffinate stream, and wherein isomerizing the hydrocarbon feed comprises isomerizing the hydrocarbon feed and the recycle stream.

13. The method of claim 10, further comprising combining the hexane-depleted raffinate stream and the isomerate product stream.

14. A method of separating a linear hexane stream from a hydrocarbon feed comprising unbranched C₄ to C₇ hydrocarbons, the method comprising:
isomerizing the hydrocarbon feed in the presence of hydrogen and in an isomerization stage comprising an isomerization catalyst to produce an isomerized hydrocarbon stream comprising branched hydrocarbons and linear hexane;
separating the isomerized hydrocarbon stream into at least an isomerate product stream comprising branched hydrocarbons and a hexane-containing raffinate stream comprising linear hexane in an isomerate separation stage in fluid communication with the isomerization stage;
splitting the hexane-containing raffinate stream into a recycle stream and a recovery stream;
separating the linear hexane stream from the recovery stream in a hexane separation stage different from and in fluid communication with the isomerate separation stage to produce the linear hexane stream and a hexane-depleted raffinate stream;
returning the recycle stream to the isomerization stage; and
isolating the linear hexane stream as an independent product stream;
wherein the linear hexane stream contains less than or equal to about 3 ppm benzene; and
returning the hexane-depleted raffinate stream to the isomerization stage, and wherein isomerizing the hydrocarbon feed comprises isomerizing the hydrocarbon feed, the recycle stream, and the hexane-depleted raffinate stream.

15. (canceled)

16. The method of claim 14, wherein the isomerate separation stage comprises an adsorption unit that preferentially adsorbs linear hydrocarbons over branched and cyclic hydrocarbons, wherein the hexane-containing raffinate stream further comprises linear pentane, and wherein separating the isomerized hydrocarbon stream comprises adsorbing linear pentane and linear hexane from the isomerized hydrocarbon stream to form the hexane-containing raffinate stream separate from the isomerate product stream.

17. The method of claim 16, wherein the hexane separation stage comprises a second fractionation unit, and wherein separating the linear hexane stream comprises fractionating the hexane-containing raffinate stream into the hexane-depleted raffinate stream as an overhead pentane stream and the linear hexane stream as a second fractionation bottoms stream.

18. The method of claim 14, wherein the isomerate separation stage comprises a fractionation unit, wherein the hexane-containing raffinate stream comprises linear hexane, cyclic hydrocarbons, and monomethyl-branched pentane, wherein the isomerate product stream comprises branched hydrocarbons having less than or equal to 6 carbon atoms and linear hydrocarbons having less than or equal to 5 carbon atoms, and wherein separating the isomerized hydrocarbon stream comprises fractionating the isomerized hydrocarbon stream into the isomerate product stream in a fractionation overhead, a fractionation bottoms stream, and the hexane-containing raffinate stream in a fractionation side draw.

19. The method of claim 18, wherein the hexane separation stage comprises an adsorption unit that preferentially adsorbs linear hydrocarbons over branched and cyclic hydrocarbons, and wherein separating the linear hexane stream comprises adsorbing the linear hexane from the at least a portion of the hexane-containing raffinate stream to produce the linear hexane stream and the hexane-depleted raffinate stream comprising cyclic hydrocarbons and monomethyl-branched pentane.

20. An isomerization apparatus comprising:
an isomerization unit for receiving a hydrocarbon feed comprising unbranched C₄ to C₇ hydrocarbons and for isomerizing the hydrocarbon feed to produce an isomerized hydrocarbon stream;
an isomerate separation unit in fluid communication with the isomerization unit for receiving the isomerized hydrocarbon stream and for separating the isomerized hydrocarbon stream into an isomerate product stream and a hexane-containing raffinate stream; and
a linear hexane separation unit in fluid communication with the hexane-containing raffinate stream from the isomerate separation unit for receiving the hexane-containing raffinate stream, for separating a linear hexane stream from at least a portion of the hexane-containing raffinate stream, and for isolating the linear hexane stream as an independent product stream.
